(19) United States
(12) Patent Application Publication
(10) Pub. No.: US 2023/0078211 A1
(43) Pub. Date: Mar. 16, 2023

(54) DECONTAMINATION SYSTEM

(71) Applicants: CASE WESTERN RESERVE UNIVERSITY, Cleveland, OH (US); The Cleveland Clinic Foundation, Cleveland, OH (US)

(72) Inventors: Jacob G. Scott, Cleveland, OH (US); Ian Charnas, Cleveland, OH (US)

(21) Appl. No.: 17/561,778

(22) PCT Filed: Mar. 17, 2021
(86) PCT No.: PCT/US21/22711

§ 371 Date: Dec. 24, 2021

Related U.S. Application Data
(60) Provisional application No. 63/035,201, filed on Jun. 5, 2020.
(60) Provisional application No. 63/067,687, filed on Aug. 19, 2020.

(51) Int. Cl. A61L 2/10; A61L 2/24

(52) U.S. Cl. CPC A61L 2/10; A61L 2/24; A61L 2202/122; A61L 2202/121; A61L 2202/11; A61L 2202/14; A61L 2202/24

(57) ABSTRACT

A decontamination system includes a housing having at least one inner surface defining a chamber. An irradiation system supplies ultraviolet germicidal irradiation (UVGI) to the chamber at a level effective to at least one of inactivate SARS-CoV-2 or inactivate at least 90% single-stranded RNA viruses present on an object positioned in the chamber in less than about 2 minutes.

### RELATED APPLICATIONS

This application claims priority from U.S. Provisional Application Ser. No. 63/035,201, filed Jun. 5, 2020 and 63/067,687, filed Aug. 19, 2020, the entirety of which are incorporated herein by reference.

### TECHNICAL FIELD

The present invention relates generally to decontamination systems and, more specifically, relates to a portable, rapid decontamination system for medical garments and supplies.

### BACKGROUND

Personal protective equipment (PPE) is essential for protecting medical personnel and patients during outbreaks of infectious disease. In particular, the use of face shields, surgical masks, and N95 respirators are recommended for infections that may be transmitted by respiratory droplets or airborne particles. Due to the rapidly emergent nature of the novel coronavirus disease (COVID-19) and stringent requirements of proper PPE protocols, many hospitals are running dangerously low on these protective devices. As a result, both patients and their healthcare providers are at increased risk of contracting and spreading SARS-CoV-2, the virus responsible for COVID-19.

One method of preserving the current supply of PPE is through cycles of decontamination and re-use with ultraviolet germicidal irradiation (UVGI). Substantial work has been done to evaluate the safety and efficacy of UVGI for decontamination of N95 filtering faceplate respirators (FFRs). Recently, UVGI has also been used to facilitate decontamination and re-use of plastic face shields. High energy UV-C rays can damage DNA and RNA via cross-linking of thymidine and uracil nucleotides, respectively, thereby preventing the replication of microbes, such as bacteria and viruses.

### SUMMARY

In one example, a decontamination system includes a housing having at least one inner surface defining a chamber. An irradiation system supplies ultraviolet germicidal irradiation (UVGI) to the chamber at a level effective to at least one of inactivate SARS-CoV-2 or inactivate at least about 90%, at least about 92%, at least about 94%, at least about 96%, at least about 98% or at least about 99% of single-stranded RNA viruses present on an object positioned in the chamber in less than about 2 minutes.

In another example, a decontamination system includes a housing having at least one inner surface defining a chamber. The inner surface has a reflective element configured to reflect at least about 75%, at least about 80%, at least about 85%, at least about 90% or at least about 95% of UV-C radiation. An irradiation system supplies UVGI to the chamber at a level effective to at least one of inactivate SARS-CoV-2 or inactivate at least about 90%, at least about 92%, at least about 94%, at least about 96%, at least about 98% or at least about 99% of single-stranded RNA viruses present on the object.

In another example, a decontamination system includes a housing extending from a first end to a second end and having at least one inner surface defining a chamber. The inner surface has a reflective element configured to reflect at least about 75%, at least about 80%, at least about 85%, at least about 90% or at least about 95% of UV-C radiation. A single, automated door is connected to the housing for accessing the chamber to position an object in and remove from the chamber. An irradiation system supplies UVGI to the chamber at a level effective to at least one of inactivate SARS-CoV-2 or inactivate at least about 90%, at least about 92%, at least about 94%, at least about 96%, at least about 98% or at least about 99% of single-stranded RNA viruses present on the object. The irradiation system includes UV-C bulbs arranged in rows and extending the entire length of the chamber. A cooling fan directs air along a path extending between and parallel to the bulbs from the first end to the second end of the housing. A MERV-13 filter or higher is provided in the path between the UV-C bulbs and the second end of the housing.

In another example, a method of decontaminating an object in a chamber of a housing includes positioning the object within the chamber through an automated door biased towards an open condition. The door is held in a closed condition with a latch. A decontamination cycle is performed by supplying UVGI to the chamber at a level effective to at least one of inactivate SARS-CoV-2 or inactivate at least 90% of single-stranded RNA viruses present on the object. The latch is automatically released when the decontamination cycle is complete such that the door automatically returns to the open condition to access the chamber. The decontaminated object is removed from the chamber through the opened door.

In another example, a method of decontaminating an object in a chamber of a housing includes positioning the contaminated object within the chamber through an automated door. A decontamination cycle is performed to neutralize or remove at least one contaminant from the object. The door is automatically opened when the decontamination cycle is complete. The decontaminated object is removed from the chamber through the opened door.

In another example, a decontamination system includes a housing extending from a first end to a second end and having at least one inner surface defining a chamber. An irradiation system supplies UVGI to the chamber at a level effective to at least one of inactivate SARS-CoV-2 or inactivate at least 90% of single-stranded RNA viruses present on the object. A cooling fan directs air along a path extending between and parallel to the bulbs from the first end to the second end of the housing. A MERV-13 filter or higher is provided in the path between the UV-C bulbs and the second end of the housing.

In another example, a decontamination system includes a housing having at least one inner surface defining a chamber. An irradiation system supplies ultraviolet germicidal irradiation (UVGI) to the chamber at a level effective to at least one of inactivate SARS-CoV-2 or inactivate at least 90% of single-stranded RNA viruses present on an object positioned in the chamber in a cycle time less than about 2 minutes. A controller controls operation of the irradiation system. A user interface is connected to the controller and allows a user to set the cycle time.

In another example, a decontamination system includes a housing having at least one inner surface defining a chamber. An irradiation system supplies ultraviolet germicidal irradiation (UVGI) to the chamber at a level effective to at least one of inactivate SARS-CoV-2 or inactivate at least 90% of single-stranded RNA viruses present on an object positioned in the chamber in less than about 2 minutes. A sensor is provided for detecting UV-C radiation levels within the chamber and has a mask partially transparent to UV-C radiation.

In another example, a decontamination system includes a housing having at least one inner surface defining a chamber. An irradiation system is provided for performing a decontamination cycle by supplying ultraviolet germicidal irradiation (UVGI) to the chamber at a level effective to at least one of inactivate SARS-CoV-2 or inactivate at least 90% of single-stranded RNA viruses present on an object positioned in the chamber. A single, automated door is connected to the housing and is biased towards an open condition for placing the object in the chamber. The door is held in a closed condition by a latch during the decontamination cycle with the latch being automatically released when the decontamination cycle is complete such that the door automatically returns to the open condition to access the chamber and remove the decontaminated object.

In another aspect, taken alone or in combination with any other aspect, the irradiation system is effective to at least one of inactivate SARS-CoV-2 or inactivate at least about 90% single-stranded RNA viruses present on the object in less than about 1 minute.

In another aspect, taken alone or in combination with any other aspect, the irradiation system is configured to deliver at least about 2 J·cm⁻² of UV-C radiation to all exterior surfaces of the object in less than about 1 minute.

In another aspect, taken alone or in combination with any other aspect, the inner surface is provided with a reflective element that reflects more than about 75% of UV-C radiation back to the object.

In another aspect, taken alone or in combination with any other aspect, the reflective element comprises porous expanded polytetrafluoroethylene (ePTFE).

In another aspect, taken alone or in combination with any other aspect, the irradiation system comprises UV-C bulbs arranged in rows and extending the entire length of the chamber.

In another aspect, taken alone or in combination with any other aspect, a cooling fan directs air along a path extending between and parallel to the bulbs from a first end to a second end of the housing.

In another aspect, taken alone or in combination with any other aspect, a MERV-13 filter or higher is provided in the path between the UV-C bulbs and the second end of the housing.

In another aspect, taken alone or in combination with any other aspect, the irradiation system comprises UV-C bulbs arranged in rows and extending the entire length of the chamber and the inner surface is provided with a reflective element comprising porous ePTFE that reflects more than 75% of UV-C radiation back to the object such that the irradiation system is configured to deliver at least 2 J·cm⁻² of UV-C radiation to all exterior surfaces of the object in less than about 1 minute.

In another aspect, taken alone or in combination with any other aspect, the housing includes a single, automated door for accessing the chamber to position the object in and remove from chamber.

In another aspect, taken alone or in combination with any other aspect, a sensor detects UV-C radiation levels within the chamber and includes a mask partially transparent to UV-C radiation.

In another aspect, taken alone or in combination with any other aspect, the mask comprises a cellophane mask.

In another aspect, taken alone or in combination with any other aspect, a controller is provided for controlling operation of the irradiation system. A user interface is connected to the controller and allows a user to program a decontamination cycle duration.

In another aspect, taken alone or in combination with any other aspect, the object comprises a face shield, respirator, or surgical mask.

In another aspect, taken alone or in combination with any other aspect, the irradiation system is configured to provide at least a 3.0-log reduction in single-stranded RNA viruses present on the object.

In another aspect, taken alone or in combination with any other aspect, wherein the door automatically opens for positioning the contaminated object within the chamber.

In another aspect, taken alone or in combination with any other aspect, the door is biased towards an open condition and held in a closed condition with a latch during decontamination. The latch is released when the decontamination cycle is complete such that the door automatically returns to the open condition to access the chamber.

In another aspect, taken alone or in combination with any other aspect, the mask comprises a cellophane mask.

Other objects and advantages and a fuller understanding of the invention will be had from the following detailed description and the accompanying drawings.

### BRIEF DESCRIPTION OF THE DRAWINGS

FIG. 1 is a schematic illustration of an example decontamination system.

FIG. 2A is a front view of the decontamination system with a door and front panel removed.

FIG. 2B is a section view taken along line 2B-2B of FIG. 1.

FIG. 3 is an enlarged view of a portion of FIG. 2B.

FIG. 4 is a section view taken along line 4-4 of FIG. 1.

FIG. 5 is a schematic illustration of a controller for the decontamination system.

FIG. 6 is a schematic illustration of a sensor for the decontamination system.

FIG. 7 illustrates the ability of the decontamination system to sanitize different portions of PPE for different pathogens.

FIG. 8 illustrates the ability of the decontamination system to reduce MS2 virus loads on contaminated masks.

### DETAILED DESCRIPTION

The present invention relates generally to decontamination systems and, more specifically, relates to a portable, rapid decontamination system for medical garments and supplies. In one example, the decontamination system includes UV-C irradiation bulbs extending the length of the decontamination chamber. The chamber is lined with a highly UV-C reflective, e.g., at least about 75%, at least about 80%, at least about 85%, at least about 90% or at least about 95% UV-C reflective, surface that greatly facilitates irradiation of the entire exterior of contaminated objects placed within the chamber.

The decontaminated objects are suspended within the chamber by UV-C transmissive support members that prevent irradiation shadowing and contribute to rapid, thorough decontamination. The decontamination system is capable of delivering at least about 2 J·cm⁻² of UV-C radiation to the decontamination chamber in under about 1 minute. Consequently, the decontamination system delivers irradiation at a level effective to at least one of inactivate SARS-CoV-2 or inactivate at least about 90%, at least about 92%, at least about 94%, at least about 96%, at least about 98% or at least about 99% of single-stranded RNA viruses present on an object positioned in the chamber in less than about 2 minutes. Depending on the type of object (porous versus non-porous, etc.) and contaminant to be neutralized or removed therefrom, the decontamination system can neutralize or remove the contaminants in as quick as about less than 30 seconds.

The decontamination system is configured to be lightweight and portable. To this end, the decontamination system can be sized to fit on a standard examination table or nursing station. In one example, the decontamination system is about 70 cm or less, about 60 cm or less or about 50 cm or less in height, less than about 30 cm, less than about 20 cm or less than about 15 cm wide, and less than about 30 cm, less than about 20 cm or less than about 15 cm deep. Consequently, the irradiation chamber can be less than about 0.065 m², less than about 0.050 m², less than about 0.035 m², less than about 0.020 m² or less than about 0.010 m².

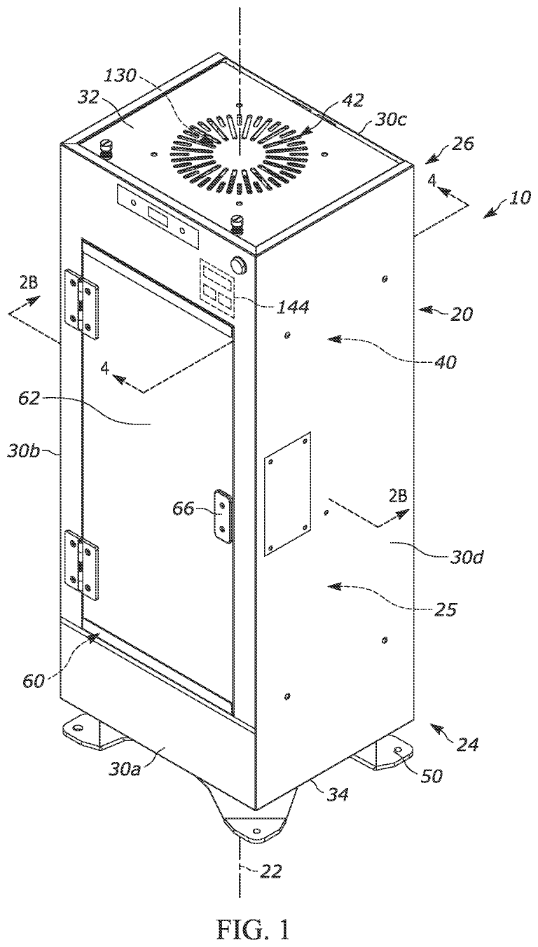

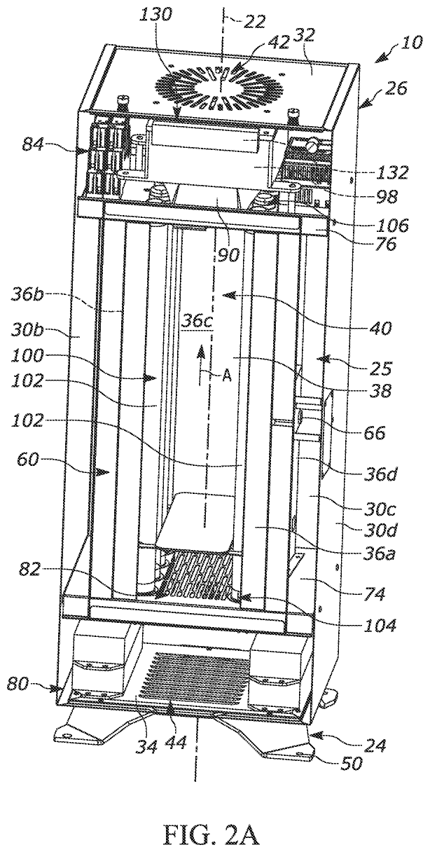

FIGS. 1-5 illustrate an example decontamination system 10. Referring to FIGS. 1-2A, the system 10 includes a housing 20 extending generally along a centerline 22 from a first end 24 to a second end 26. The housing 20 includes at least one outer wall 30, a top 32, and a bottom 34 that cooperate to define an interior space 25. As shown, the housing 20 is rectangular and therefore has four walls 30 (indicated at 30a, 30b, 30c, 30d for clarity). Other shapes for the housing 20, including cylindrical, are contemplated and, thus, the housing may only have a single wall 30. The walls 30(a-d), top 32, and bottom 34 can be formed from individual sheets (as shown) or integrally formed with one another (not shown). In any case, the housing 20 also includes one or more interior or inner walls 36 (three inner walls 36a-36d are shown). Each inner wall 36 has an inner surface 38 helping to define a primary or main chamber 40. The inner walls 36(a-d) can be formed from individual sheets (as shown) or integrally formed with one another (not shown).

One or more openings 42 extend through the top 32. One or more openings 44 extend through the bottom 34. Feet 50 are provided on or secured to the bottom 34. The feet 50 can be provided with wheels (not shown).

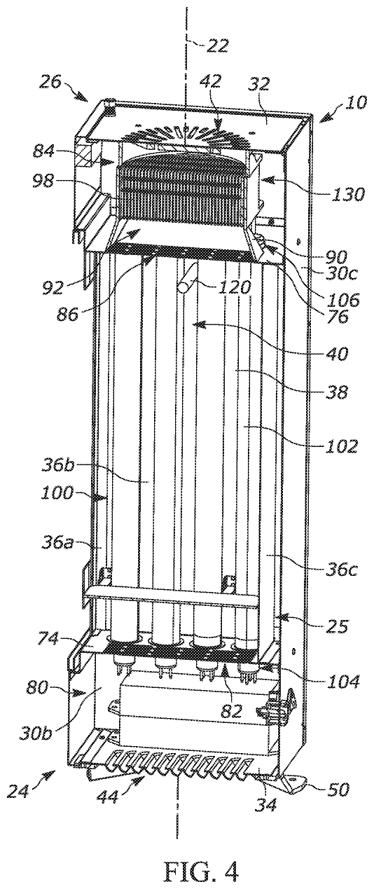

An opening 60 extends through one of the walls 30a (the front as shown) to the chamber 40. A door 62 is pivotally connected to the wall 30a for selectively closing the opening 60 and accessing the primary chamber 40. The door 62 includes a lock 66 for holding the door closed. In one example, the lock 66 includes a magnetic sensor and solenoid latching mechanism (not shown). The lock 66 can hold the door 62 shut against the bias of a spring (not shown). In such configurations, the door 62 automatically opens when the lock 66 is unlatched. The lock 66 can be connected to a controller 70 (see FIG. 5) for controlling latching and unlatching of the lock.

In another example, a proximity sensor (not shown) can be provided in the door 62 or wall 30a for allowing the user to open the door in a hands-free manner prior to using the decontamination system 10. To this end, the user can wave a hand over the proximity sensor, which sends a signal to the controller 70 to unlatch the lock 66 and open the door 62, allowing the user to then access the primary chamber 40 and use the decontamination system 10.

As shown in FIGS. 2A-2B, first and second brackets or panels 74, 76 are secured to the walls 30a-30d and the walls 36a-36c and close opposite ends of the primary chamber 40. The first panel 74 cooperates with the walls 30a-30d and bottom 34 to define a lower chamber 80 below the primary chamber 40. One or more openings 82 extend through the first panel 74 and are generally aligned with the openings 44 in the bottom 34. The lower chamber 80 fluidly connects the openings 44 to the openings 82 in the first panel 74.

The second panel 76 cooperates with the walls 30a-30d and top 32 to define an upper chamber 84 above the primary chamber 40. One or more openings 86 extend through the second panel 76 and are generally aligned with the openings 42 in the top 32. The upper chamber 84 fluidly connects the openings 42 to the openings 86 in the second panel 76.

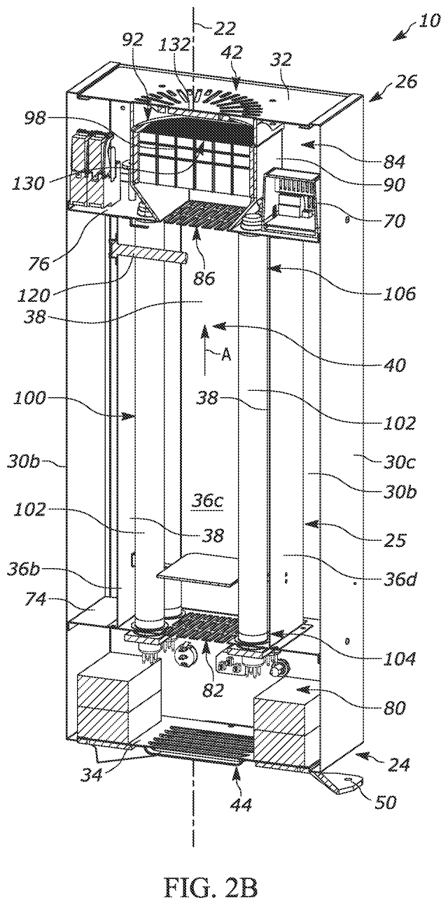

A chute or shroud 90 provided in the upper chamber 84 (see also FIG. 3) extends the entire length thereof between the second panel 76 and the top 32. The shroud 90 defines a passage 92 aligned with and extending from the openings 42 to the openings 86 in the second panel 76. Consequently, a fluid path exists from the area beneath/around the first end 24 of the housing 20, through the openings 44 in the bottom 34, through the lower chamber 80 and openings 82, into the primary chamber 40, through the openings 86 in the second panel 76, through the passage 92, and finally exiting the second end 26 of the housing at the openings 42 in the top 32.

A filter 98 is provided in the shroud 90. In one example, the filter 98 has a Minimum Efficiency Reporting Value (MERV) effective to prevent release of a contaminant from the chamber 40. For example, the filter can be a MERV-13 filter or higher, e.g., a MERV-14 filter, a MERV-15 filter, etc. In any case, the filter 98 is positioned in the flow path between the primary chamber 40 and the top 32 of the housing 20.

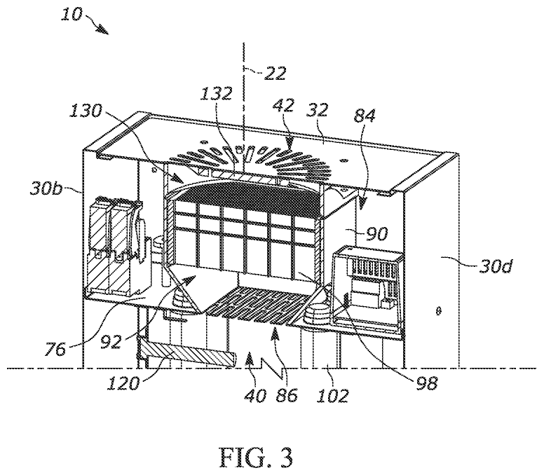

An irradiation system 100 (see also FIG. 4) is provided within the primary chamber 40 for supplying ultraviolet germicidal irradiation (UVGI), e.g., UV-C light, to the primary chamber. To this end, the irradiation system 100 includes a series of bulbs 102 that extend along/parallel to the length of the housing 20. In particular, each bulb 102 extends from a first end 104 to a second end 106. The first ends 104 of the bulbs 102 are received in openings in the first panel 74. The second ends 106 of the bulbs 102 are received in openings in the second panel 76. Consequently, each UV-C bulb 102 extends the entire length of the primary chamber 40. As shown, eight bulbs 102 are utilized, although more or fewer bulbs can be provided in the irradiation system 100. The bulbs 102 can be arranged in rows on opposite sides of the primary chamber 40.

At least one of the inner surfaces 38 of the walls 36a-36c can be formed from or provided with a reflective element for reflecting light irradiated by the bulbs 102. In one example, the reflective element comprises a material, e.g., a polymeric material such as porous expanded polytetrafluoroethylene (ePTFE), aluminum or inorganic pigments, capable of reflecting at least about 75%, at least about 80%, at least about 85%, at least about 90% or at least about 95% nominal reflectance, preferably at least about 97% nominal reflectance, of UV-C light. It will be appreciated that a desired percentage of the inner surface(s) 38 and/or a desired percentage of the entire surface area of the inner surface(s) can be provided with the reflective element. For example, greater than about 50%, greater than about 75% or 100% of the cumulative inner surface 38 surface area can be provided with the reflective element.

At least one support member 120 is provided in the primary chamber 40 and can be connected to one or more of the walls 36a-36d. The support members 120 help to suspend contaminated objects (not shown) within the primary chamber 40 and away from the walls 36a-36d and panels 74, 76. The contaminated objects can be, for example, medical supplies and/or PPE, such as surgical masks, face shields, and/or respirators. The support members 120 can be made from a material(s) that is highly transmissive of light in the UV-C spectrum, e.g., greater than about 80%, greater than about 90%, or greater than about 95% transmissive. In one instance, the support members 120 are made from fused quartz and are formed as cylindrical projections extending from the wall 36c into the primary chamber 40. Due to the aforementioned features, the irradiation system 100 is capable of delivering/supplying more than about 1 J·cm⁻², preferably more than 2 J·cm⁻², of UV-C radiation in under about 2 minutes, preferably under about 1 minute to all external surfaces of a contaminated object positioned in the chamber 40.

The decontamination system 10 further includes a cooling system 130 for helping to cool the bulbs 102 during operation thereof. The cooling system 130 includes a fan 132 provided in the upper chamber 84 to draw cooling air through the openings 44 in the bottom 34 (which act as intake/inlet openings), the openings 82, 86 in the panels 74, 76, the shroud 90, and the openings 42 in the top 32 (which act as outlet/exhaust openings).

The output of the bulbs 102 is highly dependent on bulb temperature and, thus, actively cooling the bulbs during operation can help prevent temperature-mediated decreases in UV irradiance. In one example, the bulbs 102 have a normal operating temperature range of about 25-80° C. Consequently, the cooling system 130 can include one or more temperature sensors or thermostats 140 (see FIG. 5) connected to the controller 70 for monitoring the temperature of the primary chamber 40 and, thus, monitoring the bulb 102 temperature.

The controller 70 can also be connected to an alert 142 that provides audio and/or visual notifications when, for example, the door 62 is not properly closed, one or more of the bulbs 102 is not properly connected or below a predetermined temperature, the fan 132 is not functioning, etc.

A user interface 144 can be connected to the controller 70 for allowing a user, e.g., medical personnel cleaning the objects, to control operation of the decontamination system 10. The user interface 144 can be integrated into the housing (see phantom in FIG. 1) or provided on a mobile device, e.g., smartphone or other computing device (not shown), and wirelessly connected to the controller 70 to enable remote control thereof. The user interface 144 can include knobs, dials, buttons, a keypad, etc. for allowing the user to control and program various aspects of the decontamination system 10 prior to each decontamination cycle.

One or more light sensors 150 can be connected to the controller 70 and provided in the main chamber 40 for detecting/monitoring the light output of the bulbs 102. Monitoring the light output can serve multiple purposes, including, for example, helping ensure the irradiation system 100 outputs a sufficient amount of UV-C light, and helping identify when one or more bulbs 102 need inspection or replacement.

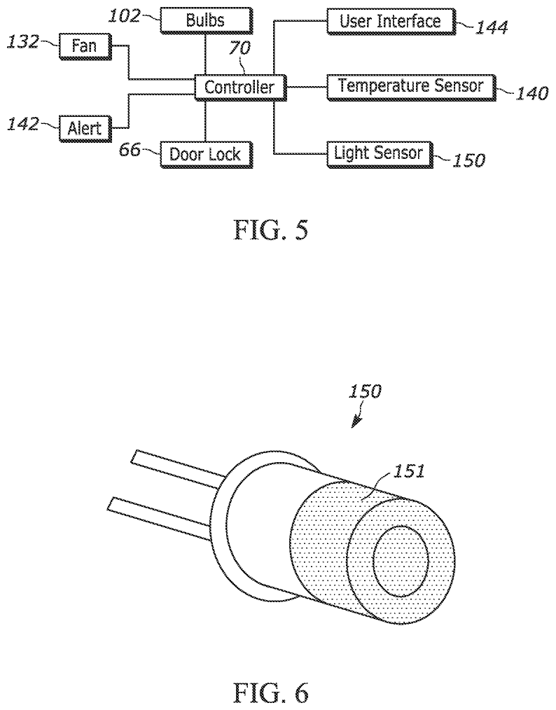

Most commercially available UV-C sensors measure up to about 20-40 mW/cm². Since the decontamination system 10 outputs UV-C irradiation at greater levels than that, a more robust light sensor is needed. That said, in one example, the light sensor 150 is attenuated with an optically-clear material having partial transparency in the UV-C range. To this end, one or more layers 151 of plastic, e.g., cellophane, are provided over the sensor 150 to help ensure the received luminescence is within the sensor operating range. One example configuration for the sensor 150 is illustrated in FIG. 6.

In operation, the user (in most cases a medical professional such as a nurse or doctor) interacts with the user interface 144 to program the decontamination system 10, i.e., pre-select operating conditions for the decontamination cycle. This can include, for example, programming power output, cycle time, number of cycles, use of or degree of active cooling, etc., depending on the type of object to be decontaminated.

The door 62 is opened—either manually or via the proximity sensor—to access the primary chamber 40. The user then hangs each contaminated object from one of the support members 120. The user then closes the door 32, which causes the controller 70 to automatically actuate the lock 66, thereby sealing the objects inside. The controller 70—via the user interface 144—can be configured to prevent operation of the decontamination system 10 as long as the door 62 is open. To this end, the controller 70 receives signals from the lock 66 and will not activate the bulbs 102 until/unless the lock is latched.

Once the door 62 is latched shut, the controller 70 automatically begins the decontamination process according to the pre-selected conditions without additional user input. More specifically, the controller 70 immediately energizes the bulbs 102 to irradiate UV-C light within the primary chamber 40. A portion of the irradiated light directly impinges on, and is absorbed by, the objects. Other portions of the irradiated light are reflected by the reflective elements on the inner surfaces 38 back toward the objects. This advantageously helps to decontaminate all/substantially all of the objects by ensuring all exterior surfaces of the objects are exposed to the desired irradiation level for the desired time period.

By providing a reflective element with such a high reflectance, the ability of the decontamination system 10 to adequately decontaminate all exterior surfaces of the objects is increased. In other words, both/all sides of the object can be contaminated simultaneously. This alleviates the need to physically move or flip the object during the decontamination process. Furthermore, using UV-C transmissive support members 120 helps to ensure any portions of the object contacting the support member, e.g., head straps for FFR, are properly exposed to the UV-C light without shadowing.

During decontamination, the controller 70 continuously receives signals from the thermostat 140. If the monitored temperature within the primary chamber 40 exceeds a predetermined threshold, e.g., above about 80° C., the controller 70 activates the fan 132 to draw cooling air into the primary chamber 40 and over the bulbs 102 in the manner indicated generally by the arrow A in FIG. 2A. More specifically, the fan 132 draws in relatively cooler air from outside the housing 20 through the inlet openings 44. The cooler air flows upwards through the lower chamber 80, through the openings 82 in the first panel 80, and into the primary chamber 40 where it cools the bulbs 102. To this end, the cooling air flows between and around the bulbs 102 while flowing upwards in the direction A.

The now heated air flows through the openings 86 in the second panel 76 and into the passage 92 in the shroud 90. Since the cooling air passes through the primary chamber 40 where decontamination of the objects occurs, it is desirable to prevent/limit any viral or contamination particles entrained in the cooling air from exiting the system 10. That said, the filter 98 in the passage 92 of the shroud 90—being downstream of the bulbs 102 and upstream of the exhaust openings 42—helps to remove viral particles from the cooling air before the air exits the system 10 through the exhaust openings. The controller 70 turns off the fan 132 when the monitored temperature falls below the predetermined threshold.

Once one complete cycle of the decontamination process finishes, the controller 70 turns off the bulbs 102 and automatically unlatches the lock 66, which automatically opens the spring-loaded door 62. This advantageously alleviates the need for the user to physically contact the door 62 to access the now decontaminated objects. That said, the primary chamber 40 is now readily accessible and the user can remove the decontaminated objects from the respective support members 120 with limited risk of re-contamination.

The decontamination system of the present invention is advantageously configured to supply UVGI to the chamber at a level effective to at least one of inactivate SARS-CoV-2 or inactivate at least 90% of single-stranded RNA viruses present on the objects positioned within the chamber. The decontamination can be performed rapidly, e.g., under about 1 minute, and with high intensity irradiation, e.g., at least 2 J·cm⁻². The high reflectively of the inner surfaces surrounding the irradiation system, coupled with the configuration of the UV-C transmissive support members and/or bulbs extending the entire length of the primary chamber enable the decontamination system to advantageously, simultaneously decontaminate all the exterior surfaces of the objects placed within the chamber. More specifically, the irradiation system of the present invention can be configured to provide at least a 3.0-log reduction in single-stranded RNA viruses present on the object.

The decontamination system is advantageously configured to be lightweight and portable compared to other decontamination systems. To this end, the decontamination system can be sized to fit on a standard examination table or nursing station. In one example, the decontamination system is about 70 cm or less, about 60 cm or less or about 50 cm or less in height, less than about 30 cm, less than about 20 cm or less than about 15 cm wide, and less than about 30 cm, less than about 20 cm or less than about 15 cm deep. Consequently, the irradiation chamber can be less than about 0.065 m², less than about 0.050 m², less than about 0.035 m², less than about 0.020 m² or less than about 0.010 m².

It will be appreciated that features of the decontamination system of the present invention can be used in scenarios that do not utilize UV-light but nevertheless remove one or more targeted contaminants from the object(s) placed within the housing via sterilization or decontamination. This can include hydrogen peroxide and/or steam chambers used to, for example, remove biological contaminants from medical equipment and/or garments. To this end, the single, automated open and/or automated close door can advantageously grant access to any type of decontamination/sterilization chamber in a hands-free manner. Furthermore, the forced/active cooling system can be implemented in and configured to operate with any type of sterilization and/or decontamination system.

### Example

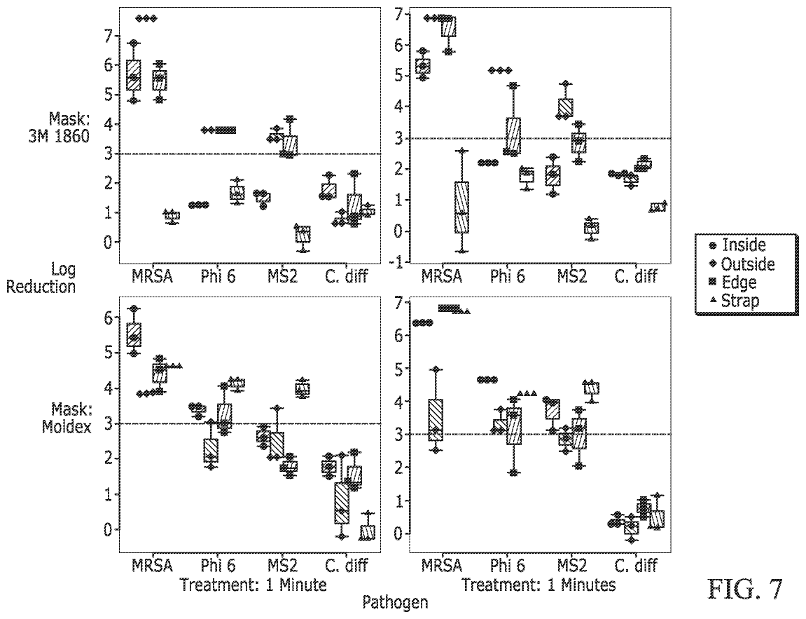

Referring to FIG. 7, we conducted pathogen load-reduction experiments to assess the ability of the decontamination system to sanitize contaminated FFRs. We tested both Moldex and 3M 1860 N95 respirators under 2 conditions representing different levels of contamination. Under condition 1, samples of Clostridioides difficile (C. diff, Escherichia virus MS2 (MS2), Psueodomnas virus phi6 (Phi 6), and methicillin-resistant Staphylococcus aureus were suspended in an 8% mucus solution. Next, 10 μL of the solution was applied in triplicate to the Moldex and 3M 1860 N95 respirators, spread 10 mm, and allowed to dry. The solution was applied to the outer surface of the mask, outer edge of the mask, inner surface of the mask, and mask strap. Following inoculation, masks were treated in the decontamination system for 1 minute or 3 minutes. Condition 1 was designed to test the ability of the decontamination system to sanitize soiled or highly contaminated masks.

Under condition 2, 1 mL of the MS2 inoculum was applied to the exterior surface of each mask in triplicate and sampling was done by swabbing the exterior of the respirator. This sampling method may mimic the risk to personnel more closely than in simulation 1 as pathogens embedded within the respirator are not detected. In simulation 2, masks were treated in the decontamination system for 1 minute only. Control masks for both simulations were inoculated following the above protocols and left untreated. Log-reduction was calculated by comparing the decontamination system-treated masks to the controls. The full experimental protocol has been previously described, including more details about inoculum and viral recovery procedures.

As expected, the reduction in pathogen load varied substantially between pathogens, but the decontamination system was highly effective at decontaminating masks soiled with MRSA. Mask location also proved to be a significant variable in pathogen load reduction. The decontamination system was most effective at reducing the levels of methicillin-resistant Staphylococcus aureus and showed moderate results in reducing the levels of Phi 6 and MS2. In addition, the decontamination system performed better on the inside, outside, and edge mask locations and all portions of the filtering device itself.

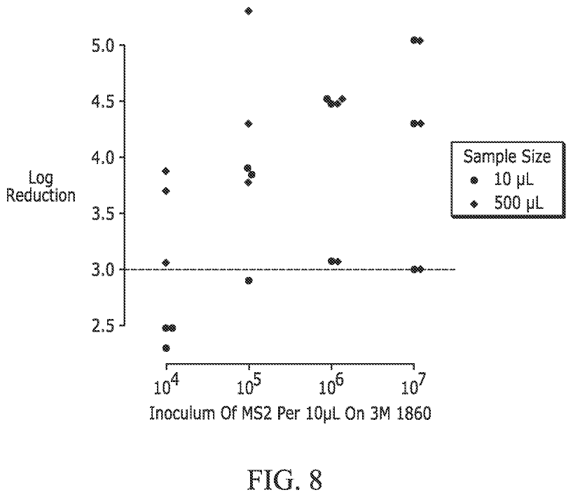

Under test condition 2, which is likely more representative of the clinical use-case, almost all experiments met or exceeded a 3-log reduction in viral recovery (FIG. 8). Indeed, the data that correspond to a less than 3-log reduction also exceeded the lower limit of detection, meaning that 0 infectious units were detected. The larger 500 mL sample volume, which improves the lower limit of detection, met or exceeded 3-log reduction in every experiment. The decontamination system of the present invention therefore consistently demonstrated a 3-log reduction in MS2 under the alternative recovery protocol in test condition 2.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

1. A decontamination system comprising:
a housing having at least one inner surface defining a chamber; and
an irradiation system for supplying ultraviolet germicidal irradiation (UVGI) to the chamber at a level effective to at least one of inactivate SARS-CoV-2, the virus responsible for COVID-19, or inactivate at least 90% of single-stranded RNA viruses present on an object positioned in the chamber in less than about 2 minutes.

2. The decontamination system of claim 1, wherein the irradiation system is effective to at least one of inactivate SARS-CoV-2 or inactivate at least 90% single-stranded RNA viruses present on the object in less than about 1 minute.

3. The decontamination system of claim 1, wherein the irradiation system is configured to deliver at least 2 J·cm⁻² of UV-C radiation to all exterior surfaces of the object in less than about 1 minute.

4. The decontamination system of claim 1, wherein the inner surface is provided with a reflective element that reflects more than 75% of UV-C radiation back to the object.

5. The decontamination system of claim 4, wherein the reflective element comprises porous expanded polytetrafluoroethylene (ePTFE).

6. The decontamination system of claim 1, wherein the irradiation system comprises UV-C bulbs arranged in rows and extending the entire length of the chamber.

7. The decontamination system of claim 6, further comprising a cooling fan for directing air along a path extending between and parallel to the bulbs from a first end of the housing to a second end.

8. The decontamination system of claim 7, further comprising a MERV-13 filter or higher provided in the path between the UV-C bulbs and the second end of the housing.

9. The decontamination system of claim 1, wherein the irradiation system comprises UV-C bulbs arranged in rows and extending the entire length of the chamber and the inner surface is provided with a reflective element comprising porous ePTFE that reflects more than 75% of UV-C radiation back to the object such that the irradiation system is configured to deliver at least 2 J·cm⁻² of UV-C radiation to all exterior surfaces of the object in less than about 1 minute.

10. The decontamination system of claim 1, wherein the housing includes a single, automated door for accessing the chamber to position the object in and remove from chamber.

11. The decontamination system of claim 1, further including a sensor for detecting UV-C radiation levels within the chamber and having a mask partially transparent to UV-C radiation.

12. The decontamination system of claim 11, wherein the mask comprises a cellophane mask.

13. The decontamination system of claim 1, wherein the object comprises one of a face shield, respirator and surgical mask.

14. The decontamination system of claim 1, wherein the irradiation system is configured to provide at least a 3.0-log reduction in single-stranded RNA viruses present on the object.

15. The decontamination system of claim 1, further comprising:
a controller for controlling operation of the irradiation system; and
a user interface connected to the controller and allowing a user to program a decontamination cycle duration.

16-28. (canceled)

29. A decontamination system comprising:
a housing extending from a first end to a second end and having at least one inner surface defining a chamber, the inner surface having a reflective element configured to reflect at least 75% of UV-C radiation;
a single, automated door connected to the housing for accessing the chamber to position an object in and remove from chamber;
an irradiation system for supplying UVGI to the chamber at a level effective to at least one of inactivate SARS-CoV-2 or inactivate at least 90% of single-stranded RNA viruses present on the object, the irradiation system comprising UV-C bulbs arranged in rows and extending the entire length of the chamber;
a cooling fan for directing air along a path extending between and parallel to the bulbs from the first end of the housing to the second end; and
a MERV-13 filter or higher provided in the path between the UV-C bulbs and the second end of the housing.

30-33. (canceled)

34. A method of decontaminating an object in a chamber of a housing, comprising:
positioning the contaminated object within the chamber through an automated door;
performing a decontamination cycle to remove at least one contaminate from the object; and
automatically opening the door when the decontamination cycle is complete; and
removing the decontaminated object from the chamber through the opened door.

35. The method of claim 34, wherein the door automatically opens for positioning the contaminated object within the chamber.

36-40. (canceled)

41. The method of claim 34, wherein the decontamination cycle is performed by supplying UVGI to the chamber at a level effective to at least one of inactivate SARS-CoV-2 or inactivate at least 90% of single-stranded RNA viruses present on the object.

42. The method of claim 34, wherein UVGI is supplied for less than about 2 minutes to decontaminate the object.
